(19) United States
(12) Patent Application Publication
(10) Pub. No.: US 2026/0315404 A1
(43) Pub. Date: Oct. 15, 2026

(54) VACCINE COMPOSITION COMPRISING A SYSTEM FOR DELIVERING AN INACTIVATED WHOLE BACTERIUM VIA CATIONIC POLYSACCHARIDE NANOPARTICLES WITHOUT ANY ADJUVANT

(71) Applicant: Vaxinano, Loos (FR)

(72) Inventors: Didier Betbeder, Loos (FR); Angélo Scuotto, Loos (FR); François Fasquelle, Loos (FR)

(21) Appl. No.: 19/156,827

(22) PCT Filed: Feb. 16, 2024
(86) PCT No.: PCT/EP2024/053960

§ 371 Date: Aug. 14, 2025

(30) Foreign Application Priority Data
Feb. 16, 2023 (FR) FR2301459
Sep. 29, 2023 (FR) FR2310463

(51) Int. Cl. A61K 9/51; A61K 39/00; A61K 39/02; A61K 39/04; A61K 39/08; A61K 39/085; A61K 39/09; A61K 39/095; A61K 39/102; A61K 39/108; A61K 39/112; A61P 37/04

(52) U.S. Cl. CPC A61K 9/5161; A61K 39/0258; A61K 39/0275; A61K 39/0283; A61K 39/04; A61K 39/08; A61K 39/085; A61K 39/092; A61K 39/095; A61K 39/102; A61K 39/105; A61P 37/04; A61K 2039/521; A61K 2039/541; A61K 2039/543; A61K 2039/552; A61K 2039/70

(57) ABSTRACT

A prophylactic vaccine composition that is intended for mammals and birds comprises a killed whole bacterium, the bacterium being covered with a cationic agent, in particular, cationic nanoparticles.

### CROSS-REFERENCE TO RELATED APPLICATIONS

This application is a national phase entry under 35 U.S.C. § 371 of International Patent Application PCT/EP2024/053960, filed Feb. 16, 2024, designating the United States of America and published as International Patent Publication WO 2024/170728 A1 on Aug. 22, 2024, which claims the benefit under Article 8 of the Patent Cooperation Treaty of French Patent Application Serial No. FR2301459, filed Feb. 16, 2023 and French Patent Application Serial No. FR2310463, filed Sep. 29, 2023.

### TECHNICAL FIELD

The present disclosure relates to the field of vaccine compositions. The present disclosure more particularly relates to a prophylactic vaccine composition that is intended for mammals and birds comprising a killed whole bacterium, the bacterium being covered with a cationic agent, in particular, cationic nanoparticles.

### BACKGROUND

Bacteria are responsible for many diseases. A single bacterial infection can be enough to induce a fatal disease, with fatal economic consequences for farms, and poultry farms, in particular. Escherichia coli is a commensal bacterium found in the digestive tract of animals and humans. This is the most common bacterium. It is found both in their environment and in their intestinal flora. Other pathogenic bacteria such as, for example, Salmonella enterica responsible for Salmonellosis, also represent a major challenge for poultry health.

To prevent the risk of infection in farm animals, prophylactic vaccines have been developed with the aim of immunizing individuals and averting the risks of infection and the resulting health consequences.

Prophylactic vaccination consists of inducing an immune response in a healthy individual who has not yet been in contact with a pathogen in order to activate their immune defenses. To achieve this, an antigen such as a pathogen or a pathogen fragment, is presented to the immune cells of the individual. This presentation activates the adaptive immune cells, the B and T lymphocytes. They multiply and produce antibodies that neutralize and eliminate the antigen and/or a cellular response that destroys infected cells. This mechanism induces an immune memory, allowing the individual to be protected the next time the pathogen is encountered. Prophylactic vaccination therefore makes it possible to immunize healthy individuals in order to protect them from future diseases.

For this mechanism to be effective, it is essential that the pathogen is identified as an intruder by the immune system of the individual so that they develop a protective immune response against the infection.

Prior art teaches us that there are several types of prophylactic vaccines for immunizing poultry against pathogenic bacteria.

European Patent Publication No. EP2911688A1 relates to a serovar of Salmonella enterica serogroup C1 for use in protecting poultry against a disorder resulting from Salmonella enterica infection. This Salmonella enterica serovar is in inactivated form. The serovar is used to manufacture vaccines that can be multivalent. Chicks are vaccinated at 30 hours of age. The serovar is used to formulate a vaccine containing an adjuvant, such as, for example, approximately 25% v/v aluminum hydroxide.

European Patent Publication No. EP0256792A2 discloses a vaccine for protecting poultry against colibacillosis infection, comprising as active ingredient E. coli cells inactivated by ultrasound treatment to destroy the bacteria. The vaccine may contain adjuvants, for example, an aluminum compound such as aluminum hydroxide gel. Inoculation of poultry with the vaccine is preferentially performed via the cloaca. However, the vaccine can also be inoculated in a conventional way, for example, intramuscularly, intravenously or subcutaneously. The essence of the invention is the use of ultrasonic cell membrane disruption in the manufacture of a vaccine against colibacillosis in poultry.

International Patent Application Publication No. WO2022/008848 discloses a process for preparing a vaccine composition from at least one lyophilized antigen comprising the steps of:
Providing an aqueous solution comprising a cationic nanoparticle consisting of a cationic polysaccharide core;
Adding the lyophilized antigen to the aqueous solution; and
Incubating the resulting composition at room temperature.

The process according to this document comprises the presence of a partial or total pathogen extract, which may contain proteins, polysaccharides and lipids. The antigen according to this document is a complex protein extract obtained from a whole pathogen.

U.S. Patent Application Publication No. US2021/093705 discloses nanoparticle compositions for use as vaccines against Salmonella enteritidis in poultry. The vaccine composition comprises: highly immunogenic protein antigens (outer membrane proteins (OMP) or whole antigenic protein (KAg)) and flagellar protein extracted from killed Salmonella enteritidis. These antigens are trapped inside polyanhydride or chitosan nanoparticles.

International Patent Application Publication No. WO2021/021778 discloses a composition comprising a polyacrylic acid mucosal adjuvant and/or inactivated antigens from respiratory or intestinal bacteria or viruses. Additionally, the composition can comprise a cell suspension comprising non-cationic polyacrylic acid particles ranging in size from 250 nm to 10 microns.

Anthony Pavic et al. describe a study concerning the development of an autologous trivalent inactivated vaccine. The trivalent vaccine was produced from equal amounts of cell suspension (3×108 CFU/mL), combined with an aluminum hydroxide adjuvant and administered to hens intramuscularly in the breast at 12 to 17 weeks of age.

The accessibility of vaccination campaigns is often limited by the cost of a dose of vaccine composition. Indeed, current vaccines are often costly, which limits their widespread use, notably in farming where the number of individuals to be treated is substantial. Although vaccines are marketed to prevent infections caused by bacteria in mammals and birds, these vaccines are unsatisfactory because they do not generate total, effective protection at an affordable cost. In addition, the compositions proposed in the prior art comprise all vaccinal adjuvants, which are necessary for their effectiveness, but whose side effects are widely documented.

### BRIEF SUMMARY

An adjuvant-free prophylactic vaccine composition has been developed for immunizing mammals and birds, particularly poultry, against pathogenic bacteria. In particular, a new delivery system has been developed, wherein the covering of at least a killed whole pathogenic bacterium with cationic nanoparticles (NPs) enhances the cellular uptake mechanism. This has the effect of improving the mechanism for presenting bacterial antigens to immune cells and therefore activating the immune system more quickly and more effectively. Advantageously, the vaccine composition can be multivalent so as to induce broad-spectrum protection. The vaccine composition can thus be used to produce combination vaccines.

Endocytosis is a mechanism by which extracellular material enters a cell by invagination of the plasma membrane followed by the formation of isolated vesicles in the cytoplasm. When endocytosis is carried out by specialized immune cells (polymorphonuclear neutrophils, macrophages, dendritic cells), reference is made to phagocytosis.

The prophylactic vaccine can be used to treat avian salmonellosis, colibacillosis, campylobacteriosis or any other bacterial infection.

Thus, the present disclosure relates to an adjuvant-free prophylactic vaccine composition, in particular, for mammals and birds, and more particularly poultry, comprising cationic nanoparticles consisting of a polysaccharide core and at least one inactivated bacterium, characterized in that the bacterium is whole and in that the cationic nanoparticles cover the bacterium. This composition limits the risks of contamination by at least one disease resulting from infection by a pathogenic bacterium. In a particular embodiment, the present disclosure relates to a multivalent prophylactic vaccine composition intended for the treatment of salmonellosis or colibacillosis notably for mammals and birds, more specifically, poultry such as laying hens, broilers, turkeys, ducks, guinea fowl, ostriches, emus, quails, etc.

The present disclosure also relates to the use of a vaccine composition to prevent a bacterial infection, in particular, colibacillosis, salmonellosis and campylobacteriosis.

Surprisingly, it has been demonstrated that the administration of a vaccine composition comprising inactivated whole pathogenic bacteria covered with cationic agents, such as, for example, cationic nanoparticles, constituted a new antigen delivery system for effective immunization against a pathogen of bacterial origin.

It is known from the prior art that cationic nanoparticles have the ability to enhance the endocytosis phenomenon of immune cells by delivering small antigens on the order of 5 to 15 nanometers in size, such as protein antigens from fragmented pathogens. In this configuration, a significant amount of nanoparticles is required to internalize each element of the fragmented bacterial cell. These delivery systems use cationic nanoparticles combined with a total or partial extract of a fragmented pathogenic bacterium, which is, in this case, contained in the core of the nanoparticles; this “antigen in the nanoparticle core” configuration requires the use of more nanoparticles than antigens (by weight), preferably 10 to 100 times more nanoparticles than antigens. This can be even higher when using PLGA nanoparticles or liposomes, for example. This prior art delivery system cannot deliver whole bacteria; it can only deliver small antigens on the order of 5 to 15 nanometers in size, whereas whole cells have a minimum size on the order of 1 to 10 microns.

In the present disclosure, it has been unexpectedly demonstrated that cationic nanoparticles allow the phenomenon of phagocytosis of whole bacterial cells. In other words, a delivery system has been developed that can deliver bacteria that are at least 100 times larger than protein antigens. This is therefore a new delivery system wherein the bacteria are covered with positively-charged nanoparticles according to the present disclosure, allowing them to interact with the cell membrane and enter the immune cells by phagocytosis. Indeed, this combination mimics the process by which viruses enter cells by virtue of the modulation of ionic charges provided by the nanoparticle. By covering a whole bacterium with nanoparticles according to the present disclosure, the “nanoparticle covering a whole bacterium” combination is positively charged. This positive charge fosters the interaction with the cell membrane and allows the whole bacterium-nanoparticle complex to enter through phagocytosis. This process is made possible by virtue of the particular combination that has been developed, namely nanoparticles covering a whole inactive bacterium. No nanoparticle-covered combination of a whole bacteria has ever been described before.

This new delivery system offers the innovative advantage of allowing a whole bacterium to enter a cell, via the vaccine composition according to the present disclosure. Advantageously, a smaller amount of nanoparticles vs. the amount by weight of bacterial proteins can be used; indeed, the amount of NPs can be 3 to 100 times fewer than that of bacterial proteins.

Thus, interestingly, even a small amount of nanoparticles is sufficient for the whole bacterial cell to enter by phagocytosis and enable effective immunization.

Reducing the number of nanoparticles used in vaccination considerably reduces the cost of producing the vaccine composition, making vaccination against pathogenic bacteria accessible on a large scale, notably in fields where cost is a real limitation to vaccination campaigns. One example is poultry farming, with in ovo vaccination.

Moreover, one advantage of the vaccine composition is that, based on the embodiment of the present disclosure, it can be multivalent. In other words, it can comprise at least two strains of bacteria, each of which is responsible for preventing infection. Thus, the present disclosure makes it easy to obtain a combined vaccine.

Based on the embodiment of the present disclosure, the vaccine composition also enables cross-immunity to be acquired. Indeed, the vaccine composition can comprise a bacterium that induces immunity against variants of the strain in question.

One application of interest for the present technology is in ovo inoculation. This approach is innovative: no previously described vaccine strategy has proposed administering an inactive whole bacterium directly into the egg. Herein, the combination of inactive whole bacteria partially covered with cationic nanoparticles proved highly effective in terms of vaccine protection, with no deleterious effect on hatching or on chicks. Moreover, by intervening before hatching, the risks of contamination within the farm (between chicks) are reduced as is the transmission of the disease within the farm. In particular, it has been shown that a vaccine composition comprising 3 different strains of E. coli, administered in ovo to chicks, can protect chicks from colibacillosis-type infection at non-lethal doses but also at lethal doses in bacterial challenge experiments. The bacterial load is reduced and the hatching rate is equivalent to that of unvaccinated eggs.

On the other hand, in another application, an intramuscular injection of a vaccine composition comprising a Salmonella bacterial strain and cationic nanoparticles (LNPs) in laying hens reduces the bacterial load and the hens lay more eggs than non-immunized hens.

The vaccine composition does not contain any adjuvants, which avoids undesirable effects. This is advantageous since the mineral adjuvants (namely mineral salts such as aluminum salts) remain in the body for a very long time. Nanoparticles act as delivery agents for killed bacteria to immune cells and induce a protective response against infection.

In the case of in ovo vaccination, the fact of not introducing molecules likely to disrupt chick development in ovo contributes to the efficacy of the vaccine approach, since the vaccine does not disrupt chick development or hatching.

The vaccine composition can be administered in ovo, but also mucosally (orally, ocularly, nasally) or intramuscularly. Furthermore, the vaccine approach according to the present disclosure can be implemented in mammals as well as in birds, notably in poultry.

The composition according to the present disclosure therefore provides a simple, easy-to-prepare and inexpensive formulation that can notably be administered in ovo. Furthermore, the fact that the composition comprises a whole and inactivated bacterium also has the advantage of being a simpler antigen to characterize than a partial or total antigen extract.

A first object of the present disclosure relates to an adjuvant-free vaccine composition comprising cationic nanoparticles consisting of a polysaccharide core and at least one inactivated bacterium, characterized in that the bacterium is whole and in that the cationic nanoparticles cover the bacterium.

In a particular embodiment, the vaccine composition is intended for mammals and birds.

In another particular embodiment, the cationic nanoparticles cover the bacteria in a bacterial protein:NP ratio by weight greater than or equal to 1. In a preferred embodiment, the bacterial protein:NP ratio by weight is greater than or equal to 2, preferably greater than 5, and even more preferably greater than 10, or even 50 or 500.

“Cationic nanoparticle consisting of a cationic polysaccharide core” means a solid nanoparticle (NP) comprising a cationic polysaccharide core. The NP may or may not be crosslinked. Its core may or may not be loaded with an anionic phospholipid. This NP is not surrounded by any phospholipid layer.

For the purposes of the present disclosure, the cationic nanoparticles are particles having a size range between 1 and 500 nanometers. More preferably, the polysaccharide nanoparticles have a size range between 10 and 300 nm, notably between 20 and 250 nm. Furthermore, a nanoparticle according to the present disclosure is advantageously used in solution. Thus, the term nanoparticle also comprises particles or molecules that are in nanoparticulate form in solution, such as, for example, chitosan and derivatives thereof. The solution may be an aqueous solution, a buffer solution or a serum solution. It has been found that certain linear molecules such as chitosan form nanometric coils in solution, which behave like conventional nanoparticles. Chitosan can thus be used as a conventional nanoparticle (e.g., Qi et al, Carbohydrate Research, 2004, 339 (16), 2693-2700) or as is or as a hydrolysate in solution.

In a first particular embodiment, the cationic polysaccharide forming the core of the nanoparticle (NP) is a non-crosslinked polymer obtained by the reaction between a polysaccharide selected from starch, dextran, chitosan, dextrin, and maltodextrin, polyfructoses (inulin), polymannoses, polygalactoses, polygalactomannans (guar gum) and at least one cationic ligand selected from a primary, secondary or tertiary amine or quaternary ammoniums. The core is not loaded with lipids. In other words, in this embodiment, the nanoparticle is a cationic nanoparticle consisting of a non-crosslinked and non-lipid-loaded polysaccharide core consisting of (i) a polysaccharide selected from starch, dextran, chitosan, dextrin and maltodextrin, polyfructoses (inulin), polymannoses, polygalactoses, polygalactomannans (guar gum) and (ii) at least one cationic ligand selected from a primary, secondary or tertiary amine or quaternary ammoniums.

In a second particular embodiment, the cationic polysaccharide forming the core of the nanoparticle (NP) is a crosslinked polymer obtained by the reaction between a polysaccharide selected from starch, dextran, chitosan, dextrin, and maltodextrin, polyfructoses (inulin), polymannoses, polygalactoses, polygalactomannans (guar gum) and at least one cationic ligand selected from a primary, secondary or tertiary amine or quaternary ammoniums, then the addition of a crosslinking agent. The crosslinking agent is selected from epichlorohydrin, a dicarboxylic acid or an acid chloride, such as sebacic acid. The core is not loaded with lipids. In other words, in this embodiment, the nanoparticle is a cationic nanoparticle consisting of a crosslinked, non-lipid-loaded polysaccharide core consisting of (i) a polysaccharide selected from starch, dextran, chitosan, dextrin, maltodextrin, polyfructoses (inulin), polymannoses, polygalactoses, polygalactomannans (guar gum) (ii) at least one cationic ligand selected from a primary, secondary or tertiary amine or quaternary ammoniums, and (iii) a crosslinking agent is selected from epichlorohydrin, a dicarboxylic acid or an acid chloride, such as sebacic acid.

In a third embodiment, the nanoparticle (NP) is a nanoparticle consisting of a phospholipid-loaded, non-crosslinked cationic polysaccharide core. In other words, in this embodiment, the nanoparticle is a cationic nanoparticle consisting of an anionic phospholipid-loaded non-crosslinked polysaccharide core consisting of (i) a polysaccharide selected from starch, dextran, chitosan, dextrin, maltodextrin, polyfructoses (inulin), polymannoses, polygalactoses, polygalactomannans (guar gum), (ii) at least one cationic ligand selected from a primary, secondary or tertiary amine or quaternary ammoniums, and (iii) an anionic phospholipid selected from diacyl phosphatidyl glycerol, diacyl phosphatidyl serine or diacyl phosphatidyl inositol.

In a fourth embodiment of the present disclosure, the nanoparticle (NP) is a cationic nanoparticle consisting of an anionic phospholipid-loaded crosslinked polysaccharide core consisting of (i) a polysaccharide selected from starch, dextran, chitosan, dextrin, and maltodextrin, polyfructoses (inulin), polymannoses, polygalactoses, polygalactomannans (guar gum), (ii) at least one cationic ligand selected from a primary, secondary or tertiary amine or quaternary ammoniums, (iii) a crosslinking agent is selected from epichlorohydrin, a diacarboxylic acid or an acid chloride, such as sebacic acid, and (iv) an anionic phospholipid selected from diacyl phosphatidyl glycerol, diacyl phosphatidyl serine or diacyl phosphatidyl inositol.

In a preferred embodiment applying to all four types of nanoparticles (NPs) described hereinbefore, the cationic polysaccharide is maltodextrin-based; it is obtained by the reaction between maltodextrin and glycidyl trimethylammonium, whether or not the NP is crosslinked or lipidated. In other words, the cationic polysaccharide core comprises maltodextrin and glycidyl trimethylammonium.

In a preferred embodiment applicable to nanoparticles (NP) with a phospholipid-loaded core, the NP is a DPPG-loaded cationic polysaccharide nanoparticle, whether or not the NP is crosslinked.

The group of birds is particularly understood to mean poultry. Poultry, for the purposes of the present disclosure, are domestic birds that serve as a source of eggs or meat and which comprise commercially important species such as, for example, chickens, laying hens, turkeys, ducks, geese, guinea fowl, pheasants, pigeons and peacocks.

In a preferred embodiment, the bacterium is selected from the following group: Salmonella enterica ser. Typhi; Streptococcus pneumoniae; Haemophilus influenzae type b; Mycobacterium tuberculosis; Extraintestinal pathogenic E. coli (ExPEC); enterotoxigenic E. coli (ETEC); S. enterica ser.; Paratyphi A; Neisseria gonorrhoeae; Clostridium difficile; Campylobacter spp; Shigella spp; Staphylobacter aureus; Helicobacter pylori.

For the purposes of the present disclosure, “whole bacteria” means unfragmented bacteria in their complete form, in particular, with their cell membrane intact. In other words, this means that membranes of the bacteria remain unaltered. On the contrary, bacteria with damaged, fragmented or exploded membranes cannot be considered to be whole bacteria for the purposes of the present disclosure.

For the purposes of the present disclosure, “cationic agent” means an agent with a positive electrical charge, such as a cationic nanoparticle.

For the purposes of the present disclosure, “inactivated or inactive bacteria” means non-living bacteria that have been previously killed but are still intact. Intact means bacteria with an unaltered membrane. They can be killed, for example, by treatment with formaldehyde or any other inactivation method known to the skilled person.

For the purposes of the present disclosure, “covered with cationic nanoparticles” means that the nanoparticles coat the surface of the inactivated bacteria. The nanoparticles cover the killed bacteria with a homogeneous layer. The coverage rate can be defined by the ratio by weight of bacterial proteins:NP. In a preferred embodiment, the ratio by weight of bacterial protein:NP is greater than or equal to 2, preferably greater than 5 and even more preferably greater than 10, or even 50 or 500.

Good efficacy of NP-covered bacteria associated with infection prevention was observed for a bacterial protein (dry weight):NP ratio by weight of between 1:0.01 and 1:10 and more particularly between 1:0.01 and 1:3. That is around 100 times fewer nanoparticles by weight with respect to the weight of the bacteria. In a particular embodiment, this ratio is greater than 1, in particular, the amount by weight of bacterial proteins is at least equal to that of NPs and can be up to 100 times greater (ratio between 1:1 and 1:0.01). In an even more preferred embodiment, the amount by weight of bacterial proteins is 2 to 100 times greater than that of NP (ratio is between 1:0.5 and 1:0.01). In an even more preferred embodiment, the amount by weight of bacterial proteins is 10 to 100 times greater than that of NP (ratio is between 1:0.01 and 1:0.1). In particular embodiments, this ratio can be between 1:0.1 and 1:10 or even between 1:0.1 and 1:3.

The vaccine composition comprises at least one bacterium to induce effective protection against, or at least reduce, a bacterial infection.

For the purposes of the present disclosure, “avoid infection” means that the vaccine composition can provide 100% protection against the risks of infection or, if it does not totally avoid the risk of infection, then the protection conferred by the vaccine is sufficient for the individual not to trigger the disease or, if it is triggered, the symptoms of the infection are at least reduced and the individual avoids death. Avoiding infection also means preventing the infection from spreading within the farm.

In one embodiment of the present disclosure, the vaccine composition is prophylactic.

For the purposes of the present disclosure, “a prophylactic vaccine composition” means a vaccine composition that makes it possible to induce an immune response in a healthy individual who has not yet been in contact with a pathogen, with the aim of activating their immune defenses and of preparing the immune system to react against a future infection.

In another embodiment of the present disclosure, the vaccine composition is intended for birds. In a particular embodiment of the present disclosure, the prophylactic vaccine composition is intended for poultry, in particular, the embryo in the egg.

For the purposes of the infection, “multivalent vaccine composition” means that the vaccine composition comprises several different bacteria enabling immunity to be induced against several diseases associated with the different bacteria.

In a particular embodiment, the vaccine composition can be used to produce combination vaccines.

For the purposes of the present disclosure, “combination vaccines” means a vaccine composition comprising several bacteria of different species or families, so as to induce immunity against several different bacteria simultaneously.

In one embodiment, the vaccine composition comprises at least one whole and inactivated bacterium to induce cross-immunity in the vaccinated individual.

For the purposes of the present disclosure, “cross-immunity” means acquired immunity against a bacterial pathogen that confers immunity against another bacterial pathogen of a different species, strain or family, which is not part of the vaccine composition.

Cross-immunity is linked to the phenomenon of cross-reactivity. Antibodies are usually specific to a particular antigen. This specificity enables antibodies to target and eliminate the antigens they have detected. A mutant bacterium conserves common antigens that can be the target of a vaccine-induced response.

Thus, cross-reactions can occur with bacteria of closely related species. Bacteria have numerous surface antigens. When an animal is immunized against a bacterium by virtue of an injection of whole bacteria, it produces antibodies against numerous bacterial antigens. If two bacteria have an identical or similar antigen, the individual will have acquired immunity to both bacteria.

In another embodiment of the present disclosure, the vaccine composition is multivalent and comprises at least 2 different strains of bacteria of different species or families, the bacteria being whole and inactivated.

The vaccine composition can, for example, be composed of 3 inactivated E. coli strains mixed with lipidated maltodextrin nanoparticles (NPs) in order to prevent colibacillosis.

Thus, according to the various embodiments of the vaccine composition, the composition may comprise:
A whole and inactivated bacterium covered with cationic nanoparticles consisting of a porous polysaccharide core in crosslinked form loaded with anionic phospholipid.
A whole and inactivated bacterium, covered with cationic nanoparticles consisting of a porous polysaccharide core in non-lipid-loaded crosslinked form.
A whole and inactivated bacterium, covered with cationic nanoparticles consisting of a polysaccharide core in non-crosslinked form loaded with anionic phospholipid.
A whole and inactivated bacterium, covered with cationic nanoparticles consisting of a polysaccharide core in non-crosslinked, non-lipid-loaded form.
At least two bacteria of different strains and/or species or families, the bacteria being inactivated and whole, covered with cationic nanoparticles consisting of a porous polysaccharide core in crosslinked form loaded with phospholipid.
At least two bacteria of different strains and/or species or families, the bacteria being inactivated and whole, covered with cationic nanoparticles consisting of a non-lipid-loaded polysaccharide core in crosslinked form.
At least two bacteria of different strains and/or species or families, the bacteria being inactivated and whole, covered with cationic nanoparticles consisting of a porous polysaccharide core in non-crosslinked form loaded with phospholipid.
At least two bacteria of different strains and/or species or families, the bacteria being inactivated and whole, covered with cationic nanoparticles consisting of a non-lipid-loaded polysaccharide core in non-crosslinked form.

A second object of the present disclosure relates to a vaccine composition as defined previously comprising at least two different inactivated bacteria, characterized in that the bacteria are whole and are covered with cationic nanoparticles consisting of a polysaccharide core. It is used in a form suitable for intramuscular, mucosal or in ovo administration. This composition is therefore multivalent and can be used to obtain a combined vaccine.

For the purposes of the present disclosure, “different inactivated bacteria” means bacteria of different strains and/or species or families.

A third object of the present disclosure relates to the use of a vaccine composition as defined previously to prevent a bacterial infection in a mammal or a bird.

In particular embodiments of the present disclosure, the bacterial infection is salmonellosis, colibacillosis or campylobacteriosis.

In a preferred embodiment, this use applies to the prevention of bacterial infection in poultry embryos in ovo (by in ovo administration).

The present disclosure also relates to a method of preventing a disease linked to a bacterial infection intended for mammalian animals and birds, comprising a vaccine composition comprising at least one inactivated whole pathogenic bacterium covered with cationic nanoparticles consisting of a polysaccharide core and comprising the following steps:
Having cationic nanoparticles consisting of a polysaccharide core and at least one whole bacterium;
Inactivating the whole bacteria with formaldehyde;
Mixing the cationic nanoparticles with the inactivated whole bacteria to obtain the vaccine composition; and
Administering the vaccine composition to the animals.

In a particular embodiment, the mixing of cationic nanoparticles with the inactivated whole bacterium is carried out so that the nanoparticles cover the bacterium in a bacterial protein:NP ratio by weight greater than or equal to 1. In a preferred embodiment, the bacterial protein:NP ratio by weight is greater than or equal to 5.

In the vaccine composition, the cationic nanoparticles may be selected from:
Cationic nanoparticles consisting of a non-crosslinked, non-lipid-loaded polysaccharide core consisting of (i) a polysaccharide selected from starch, dextran, chitosan, dextrin and maltodextrin, polyfructoses (inulin), polymannoses, polygalactoses, polygalactomannans (guar gum) and (ii) at least one cationic ligand selected from a primary, secondary or tertiary amine or quaternary ammoniums;
Cationic nanoparticles consisting of a non-lipid-loaded, crosslinked polysaccharide core consisting of (i) a polysaccharide selected from starch, dextran, chitosan, dextrin and maltodextrin, polyfructoses (inulin), polymannoses, polygalactoses, polygalactomannans (guar gum) (ii) at least one cationic ligand selected from a primary, secondary or tertiary amine or quaternary ammoniums, and (iii) a crosslinking agent is selected from epichlorohydrin, a dicarboxylic acid or an acid chloride, such as sebacic acid;
Cationic nanoparticles consisting of an anionic phospholipid-loaded, non-crosslinked polysaccharide core consisting of (i) a polysaccharide selected from starch, dextran, chitosan, dextrin and maltodextrin, polyfructoses (inulin), polymannoses, polygalactoses, polygalactomannans (guar gum), (ii) at least one cationic ligand selected from a primary, secondary or tertiary amine or quaternary ammoniums, and (iii) an anionic phospholipid selected from diacyl phosphatidyl glycerol, diacyl phosphatidyl serine or diacyl phosphatidyl inositol;
Cationic nanoparticles consisting of an anionic phospholipid-loaded, crosslinked polysaccharide core consisting of (i) a polysaccharide selected from starch, dextran, chitosan, dextrin, and maltodextrin, polyfructoses (inulin), polymannoses, polygalactoses, polygalactosemannans (guar gum), (ii) at least one cationic ligand selected from a primary, secondary or tertiary amine or quaternary ammoniums, (iii) a crosslinking agent is selected from epichlorohydrin, a diacarboxylic acid or an acid chloride, such as sebacic acid, and (iv) an anionic phospholipid selected from diacyl phosphatidyl glycerol, diacyl phosphatidyl serine or diacyl phosphatidyl inositol.

In a preferred embodiment, the cationic polysaccharide is obtained by the reaction between maltodextrin and glycidyl trimethylammonium, whether or not the NP is crosslinked.

In a particular embodiment, the prevention method comprises a vaccine composition inoculated mucosally, by injection and/or administered in ovo.

In particular embodiments of the present disclosure, the vaccine composition is administered in poultry, namely in ovo, mucosally and orally in chicks and intramuscularly in laying hens.

### BRIEF DESCRIPTION OF THE DRAWINGS

FIG. 1: E. coli uptake after covering the cells with LNP. The delivery of whole E. coli bacteria was evaluated in human H292 cells. Fluorescent FITC-E. coli, alone or combined with LNP (ratio 1:3 to 1:0.05) were incubated with human H292 cells for 4 hours, and the percentage of positive cells was measured by flow cytometry with or without Trypan blue (TB). The results represent the mean±SEM of 3 experiments. Statistical analysis: two-way ANOVA, ** p<0.01, *** p<<0.001.

FIG. 2: Intracellular delivery by confocal microscopy with a 1:3 ratio. The delivery of E. coli bacteria was evaluated in H292 cells. Fluorescent FITC-E. coli associated with LNP (ratio 1:3) were incubated with H292 cells for 4 hours, and intracellular localization was observed by confocal microscopy. A representative image was taken. Red: plasma membrane; blue: nuclei; green: E. coli. Scale bar: 10 μm.

FIG. 3: Schematic diagram of the vaccine protocol of the in ovo vaccination trial. The commercial vaccine was administered only in the positive control.

FIG. 4: Intestinal permeability test for 8 birds in each group, 6 days after the challenge (D20). The results represent the mean±SD.

FIG. 5: Analysis of anti-E. coli sIgA in the feces of 8 birds from each group, 13 days after the challenge (D27). The results represent the mean±SD of absorbance values obtained by ELISA. Statistical analysis: one-way Anova, * p<0.05.

FIG. 6: Clinical score of liver lesions from 8 birds in each group, 6 days after the challenge (D20). The results represent the mean±SD of the group scores. Statistical analysis: one-way Anova, * p<0.05.

FIG. 7: Schematic diagram of the in ovo vaccination trial protocol. Only the positive group was vaccinated on D1 with the commercial vaccine (POULVAC®).

FIG. 8: Percentage of mortality in each group after the lethal challenge at D14 (n=30).

FIG. 9: Measurement of the bacterial load in the air sacs of birds from each group, evaluated 2 days after the challenge (D16) on 8 birds, by MPN. The results represent the mean±SD. The statistical analyses were performed by one-way ANOVA, * p<0.05.

FIG. 10: Intestinal permeability of 8 birds from each group 6 days after the challenge (D20). The results represent the mean±SD. The statistical analyses were performed by one-way ANOVA, * p<0.05.

FIG. 11: Intestinal lesion score of 8 birds from each group 6 days after the challenge (D20). The results represent the mean±SD. The statistical analyses were performed by one-way ANOVA, * p<0.05.

FIG. 12: Schematic diagram of the intramuscular vaccination trial protocol.

FIG. 13: Egg laying. Top: daily number of eggs laid by hens in each group after the challenge. The error bars are hidden to clarify the graph. Bottom: Mean daily number of eggs laid by hens in each group after the challenge. Statistical analyses: one-way Anova * p<0.05, *** p<0.001, **** p<0.0001.

FIG. 14: Quantification of bacterial load in the cecum of chicken from each group, measured by qPCR. The results represent the mean±SEM. Statistical analysis: one-way ANOVA, ** p<0.01, *** p<0.001.

FIG. 15: Quantification of bacterial load in the cecum of chicken from each group, measured by qPCR. The results represent the mean±SEM.

FIG. 16: Schedule for the in ovo vaccination trial protocol. Only the “positive control” group was vaccinated on D1 with the commercial vaccine (POULVAC®).

FIG. 17: Measurement of bacterial infection in the trachea (above) and alveoli (below) of birds in each group, evaluated on 8 birds, by most probable number (MPN). The results represent the number of positive and negative birds per group.

FIG. 18: Analysis of anti-E. coli sIgA in the feces of 8 birds from each group after the challenge. The results represent the mean±SD of antibody titers. Statistical analysis: one-way Anova for each day.

FIG. 19: Lung lesion score of 8 birds per group after the challenge. The results represent the mean for each group. Statistical analyses were performed for each day by one-way ANOVA.

FIG. 20: Delivery of whole E. coli bacteria was evaluated in H292 cells. Fluorescent FITC-E. coli, alone or combined with NPs, were incubated with H292 cells for 4 hours, and the percentage of positive cells was measured by flow cytometry. The results represent the mean±SD of 2 experiments.

FIG. 21: Delivery of whole E. coli bacteria was evaluated in THP-1 cells. Fluorescent FITC-E. coli, alone or combined with NPs, were incubated with THP-1 cells for 4 hours, and the percentage of positive cells was measured by flow cytometry. The results represent the mean±SD of 2 experiments.

### DETAILED DESCRIPTION

### Examples

### Abbreviations

LNP: Crosslinked lipidated maltodextrin nanoparticles
i.d: Intradermal
i.n: Intranasal
i.p: Intraperitoneal
i.m: Intramuscular

### Example 1: Optimizing the Formulation with Cationic Nanoparticles

The aim of this study is to confirm the efficacy as a delivery system of a composition, based on a nanoparticle and an inactivated whole E. coli strain, for activating immune cells.

### 1-A Materials and Methods

### A—Vaccine Formulations

Cationic nanoparticles (LNP) are cationic lipidated maltodextrin nanoparticles.

The composition was made with an inactivated E. coli strain mixed with cationic nanoparticles. E. coli bacteria were inactivated with 0.4% formaldehyde, then purified by centrifugation. The protein content was measured by micro BCA assay. The composition was made by mixing the killed bacteria with an aqueous solution of LNP, at different weight ratios (100 μg of E. coli protein with 5, 10, 30, 50, 100 or 300 μg of LNP). The size and surface charge of the formulation were characterized respectively by dynamic light scattering (DLS) and electrophoretic light scattering (ELS) (Zetasizer NanoZS, Malvern Analytical, France), to observe whether the nanoparticles covered the surface of the killed bacteria.

B—Delivery of E. coli

The ability of LNPs to increase uptake of inactivated whole E. coli by immune cells was evaluated by flow cytometry and confocal microscopy.

Labeling E. coli with Fluorescein:

Inactivated bacteria were labeled with fluorescein isothiocyanate (FITC), by mixing 5 mg of E. coli with 50 μg of FITC (1%, Sigma, France) in sodium carbonate buffer at pH 8.3 for 2 hours. They were then dialyzed on a 10 kDa dialysis cassette (Thermo Fisher, France). The protein content was measured by micro BCA assay (Pierce, France). The labeled bacteria were then associated with LNPs at different weight ratios.

### Flow Cytometry:

H292 cell lines were seeded in 24-well plates at 50,000 cells per well until confluence. The cells were then incubated with the equivalent of 1 μg of protein alone or combined with different ratios of LNP, for 4 hours. The cells were then washed with phosphate-buffered saline (PBS), harvested with trypsin, and analyzed by flow cytometry on an Attune Nxt (Thermo Fisher, France). To distinguish intracellular bacterial delivery from membrane attachment, the cells were incubated with 40 μg/mL of Trypan blue (TB, Sigma France) to quench external FITC fluorescence.

### Confocal Microscopy

H292 cell lines were seeded in Labtek chambers (Fisher Sci., France) at 10,000 cells per well, up to confluence. The cells were then incubated with the equivalent of 1 μg of protein alone from killed whole bacteria or combined with LNPs, for 4 hours. The cells were washed, and the nucleus was stained by incubating Hoechst 33342 (Sigma, France) at 0.1 μg/mL for 5 minutes at 37° C. The cells were then washed and the plasma membrane was stained with AF-633-labeled agglutinin (WGA, Invitrogen France) at 1 μg/mL for 10 minutes at 37° C. The slides were washed again with PBS, fixed with 0.4% formaldehyde for 20 minutes, and mounted for microscopic observation (LSM 710 Zeiss, France).

### 1-B Results

### Characterization of Formulations:

TABLE 1
Characterization of the size of E. coli/LNP formulations
by dynamic light scattering (DLS) and zeta potential by
electrophoretic light scattering (ELS). Inactivated whole
E. coli were mixed with increasing amounts of LNP.

| | Z-average (nm) | PDI | Zeta potential (mV) |
|---|---|---|---|
| E. coli | 1420 | 0.08 | −4.6 |
| E. coli:LNP 1:0.05 | 2451 | 0.60 | −3.6 |
| E. coli:LNP 1:0.1 | 1125 | 0.30 | −1.6 |
| E. coli:LNP 1:0.3 | 716.9 | 0.57 | −1.1 |
| E. coli:LNP 1:0.5 | 1120 | 0.52 | +2.7 |
| E. coli:LNP 1:1 | 915.5 | 0.68 | +3.8 |
| E. coli:LNP 1:3 | 1010 | 0.75 | +14 |

DLS and ELS analyses showed that inactivated E. coli had a homogeneous size of 1.42 μm with a PDI of 0.08, and an anionic surface charge of −4.6 mV. This indicates that the bacterial structure has remained intact despite inactivation. When increasing amounts of LNP were added, the overall size did not vary, but the zeta potential increased progressively, becoming cationic from the 1:0.5 ratio. This indicates that the bacteria were progressively covered with the LNPs without any aggregation.

[symbol]

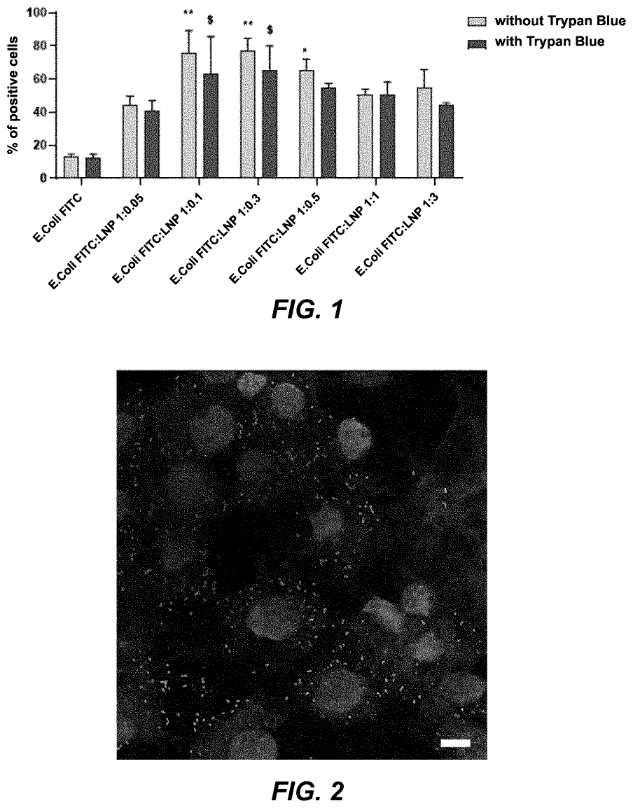

E. coli delivery by LNPs was evaluated by flow cytometry (see FIG. 1), on human airway epithelial cells (H292). Without LNP, the bacteria were endocytosed by 14% of cells. As no difference was observed in the presence of TB, this suggests that the bacteria were actually endocytosed (inside the cells). When covered with LNPs, the bacteria are absorbed by at least 40% of cells, confirming their potential as a delivery system. Moreover, delivery was significantly more efficient with a low amount of LNP, and the highest release was observed with a ratio of 1:0.3 (77%) and 1:0.1 (75.7%). In the presence of TB, 65.6% of cells were still positive with a ratio of 1:0.3 and 63% with a ratio of 1:0.1, confirming that most of the bacteria were inside the cells.

Intracellular delivery was also confirmed by confocal microscopy, with a 1:3 ratio. Around 20 to 30 E. coli bacteria (green) were observed in each cell, close to the nuclei, confirming intracellular localization.

### Conclusion:

The E. coli/LNP vaccine formulation consists of inactivated whole bacteria covered with LPN. Covering this bacterium, even with a small amount of LNP, has a significant impact on the ability of this bacteria to be taken up by cells.

### Example 2: In Ovo Vaccination Trial Against Colibacillosis

### 2-A Materials and Methods

### A—Preparation of Vaccines

The vaccine consists of 3 inactivated E. coli strains mixed with lipidated maltodextrin nanoparticles (LNP). Briefly, strains 078: K80, 01: K1, 02: K1 were inactivated with 0.4% formaldehyde, and the protein content was measured by μBCA assay. Finally, 33.3 μg per strain were mixed with LNP to obtain 100 μg of protein per vaccine dose.

### B—Animals

All animal work has been reviewed and approved by the Imunova Análises Biológicas Animal Research Ethics Committee, protocol number 06/2021.

For this experiment, 390 fertile eggs were acquired from a commercial incubator and incubated at the Imunova experimental unit. The eggs were randomly assigned to seven different experimental groups and placed in an industrial hatchery, with precise temperature and humidity control, for 21 days. The groups used in this test consisted of 30 animals and are identified in Table 1.

TABLE 2
Identification of experimental groups.

| Group | Identification | Challenge¹ |
|---|---|---|
| 1 | Negative control | — |
| 2 | Positive control | E. coli² Challenge |
| 3 | Commercial control | Mucosally vaccinated with POULVAC ® |
| | | E. coli (Zoetis) + E. coli challenge² |
| 4 | Vaccinated in ovo | Vaccinated in ovo with NP/100 μg + |
| | | E. coli challenge². |

¹All groups, including the negative control, received the live attenuated Mass I - H120 infectious bronchitis virus (IBV) vaccine orally, at a dose 100 times higher than that recommended by the manufacturer, in order to sensitize the animals to an E. coli challenge.
²Infection with 108 CFU of Escherichia coli, at a dose of 100 μL/bird orally. The challenge was confirmed by microbiological recovery of bacteria from the inoculum.

### C—Vaccination

Group 3 animals received a dose of live POULVAC® E. coli vaccine on day 1 after hatching. On the 18th day of incubation, Group 4 animals received an in ovo application of the vaccine, with a regulatory vaccine dose of 50 μL. After hatching, groups of day-old broilers were housed in isolators (1.2 m²) and fed ad libitum as recommended for their age.

### D—Challenge

At D10, all animals, including those in the negative control group, received a live attenuated Mass I-H120 infectious bronchitis virus (IBV) vaccine orally, at a dose 100 times higher than that recommended by the manufacturer.

At D14, animals in groups 2, 3 and 4 were infected orally with 10⁸ CFU of Escherichia coli (strain 19501, a different strain from that used in the vaccine), 100 μL/bird.

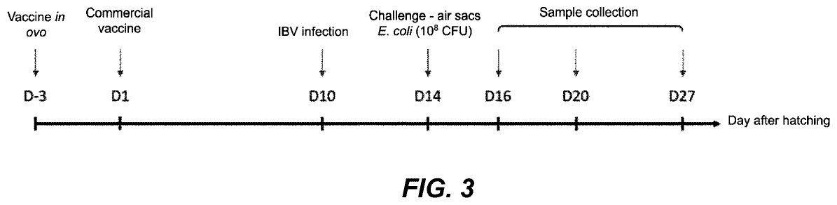

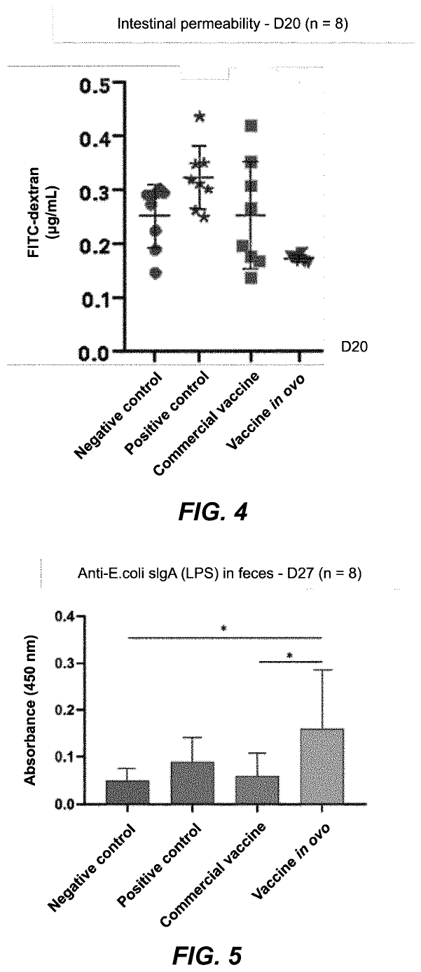

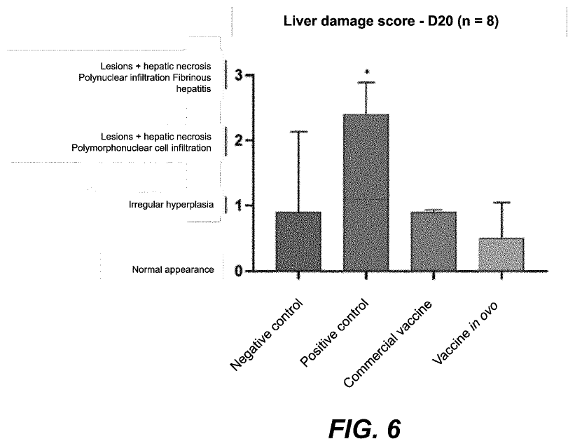

The vaccine protocol is depicted in FIG. 3.

### E—Sampling

The samples and analyses were carried out on 8 birds, at D16, D20 or D27.

### F—Analysis

### Intestinal Permeability:

Intestinal permeability was evaluated by oral administration of FITC-Dextran, a non-absorbable fluorescent marker (FITC-Dextran, 3000 to 4000 kDa), and identified in plasma/serum, to monitor gastrointestinal epithelial integrity (Vicuña et al., 2015).

### Cytokine Expression

Cytokine expression was evaluated by qPCR (IL-1β, IFNγ, IL-10, IL-4), using specific primers for each target. In this type of analysis, each combination of target and sample generates a threshold value. The Ct (threshold cycle) is a relative measure of the concentration of target-specific messenger RNA (mRNA) in the sample. This value must be normalized based on the expression of a certain reference gene, in this case, the geometric mean of the GAPDH and ACTB genes was used, generating a ΔCt value (Target Ct/Mean GAPDH+ACTB Ct) (Bustin et al., 2009). In addition to this normalization, the data were also normalized with respect to the mean ΔCt of the control group, generating a ΔΔCt (Δct/mean ΔCt Control). For undefined results, the maximum CT value (40) was considered and, for analysis purposes, was artificially modified to 41.

Quantification of Anti-E. coli sIgA:

E. coli-specific secretory IgA production was evaluated by ELISA. Briefly, the samples were diluted in 1% casein in PBS. The ELISA plates were covered with E. coli LPS (field-isolated strain). The plates were then washed three times with 200 μL/well of PBS+0.05% TWEEN®20 for 5 min/wash. The wells were blocked with 1% casein in PBS. The samples were tested in serial dilution. The plates were washed, and anti-chicken IgA was added (BioRad) diluted in 0.1% casein. After washing, the test was revealed with a solution of TMB (Life Technologies). Absorbance was read at 450 nm.

Detection and Quantification of E. coli:

Detection and quantification by MPN (Most Probable Number) of E. coli was carried out in accordance with standard ISO 7251:2005. E. coli counts were determined according to standard microbiological methods (dilution in enrichment medium followed by plating in selective/different medium). Briefly, the samples were enriched in buffered peptone water (BPW), then in EC broth and finally in EMB and MacConkey agars. The samples were serially diluted in triplicate in EPB prior to incubation, to enable quantification by the most probable number technique (Blodgett et al., 2015). For E. coli detection, only the serial dilution step has been omitted. Isolated suspect colonies were biochemically tested and confirmed.

### Liver Histology:

The birds were euthanized, and liver samples were collected and fixed according to the method of Rebel et al. (2011). The samples were embedded in paraffin and mounted on slides. All histopathological evaluations and readings were performed under the microscope by an experienced veterinary histopathologist.

TABLE 3
List of histological parameters for scoring liver damage.

| Score | Liver |
|---|---|
| 0 | Normal appearance |
| 1 | Histologically normal, sometimes with discrete focal or multifocal |
| | mononuclear hyperplasia. Absence of necrosis. |
| 2 | Possible degenerative liver damage with varying degrees of |
| | vacuolization and/or necrosis. Central or pericentral proliferative |
| | reaction involving mononuclear cells. Presence of interstitial and |
| | multifocal neutrophil infiltrate. Hyperemia/hemorrhage. |
| 3 | Same lesions as described hereinbefore, plus fibrinous perihepatitis |
| | and/or parenchymal granulomatous reactions. |

### 2-B Results

Intestinal permeability for unchallenged, unvaccinated birds (negative control) was 0.26 μg/mL, and 0.31 μg/mL for challenged, unvaccinated birds (positive control). When vaccinated with the commercial vaccine, permeability was 0.26 μg/mL, as for the negative control, confirming the efficacy of this vaccine. Furthermore, when vaccinated in ovo with the VXN-E. coli formulation, all birds showed low permeability (0.18 μg/mL), less than that of the negative control, although not significant.

Intestinal Anti-E. coli A-sIgA (LPS)

Anti-LPS secretory IgA directed against E. coli was analyzed in feces by ELISA. The absorbance obtained for the negative control birds was about 0.05 AU, and 0.095 μg/mL for the positive control birds, indicating that the oral challenge did not induce intestinal IgA secretion. Moreover, for birds vaccinated mucosally with the commercial vaccine, OD remained at 0.055 AU, as for the negative control, suggesting that this vaccine failed to promote a mucosal humoral response. In contrast, birds vaccinated in ovo with the VXN-E. coli formulation showed a significantly higher OD of 0.16 AU.

### B—Histopathology of the Liver

The liver damage score was measured 6 days after the challenge. Birds in the negative control group had a mean score of about 1, representative of discrete hyperplasia. In contrast, unvaccinated birds in the positive control group had a mean score of 2.35, suggesting liver damage and necrosis induced by E. coli infection. When vaccinated with the commercial vaccine, the mean bird lesion score was 1, as in the negative control group. Birds vaccinated in ovo had a mean lesion score<1. These results show that the commercial vaccine and in ovo vaccination protect against E. coli-induced liver damage.

### Conclusion:

This first trial indicates that in ovo vaccination with the VXN-E. coli vaccine protects birds against E. coli liver infection and induces sIgA secretion against the bacterium in the intestine.

### Example 3: In Ovo Vaccination Against a Lethal E. coli Challenge

This second trial was identical to the first in ovo trial in terms of schedule, animals per group and treatment, but with a lethal E. coli challenge. The analyses then focused on the protection provided by vaccines against bacterial load in representative organs, physiological abnormalities and subsequent mortality, observed in each group.

### 3-A. Materials and Methods

### A—Challenge

At D10, all animals, including the negative control group, received a live attenuated Mass I-H120 infectious bronchitis virus (IBV) vaccine orally, at a dose 100 times higher than that recommended by the manufacturer.

At D14, each animal in the determined group was challenged with 4.2×10¹² CFU of Escherichia coli (strain 19501), at a rate of 100 μL/bird in the air sacs.

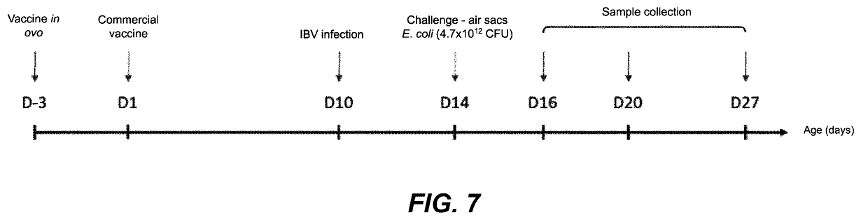

The general schedule is detailed in FIG. 7.

### B—Analysis

### Intestinal Permeability:

Intestinal permeability was evaluated by oral administration of FITC-Dextran, a non-absorbable fluorescent marker (FITC-Dextran, 3000 to 4000 kDa), and identified in plasma/serum, to monitor gastrointestinal epithelial integrity (Vicuña et al., 2015).

Detection and Quantification of E. coli:

Detection and quantification by MPN (most probable number) of E. coli was carried out in accordance with standard ISO 7251:2005. E. coli counts were determined according to standard microbiological methods (dilution in enrichment medium followed by plating in selective/different medium). Briefly, the samples were enriched in buffered peptone water (BPW), then in EC broth and finally in EMB and MacConkey agars. The samples were serially diluted in triplicate in EPB prior to incubation, to enable quantification by the most probable number technique (Blodgett et al., 2015). For E. coli detection, only the serial dilution step has been omitted. Isolated suspect colonies were biochemically tested and confirmed.

### Intestinal Histology:

The birds were euthanized, and intestinal samples were collected and fixed according to the method of Rebel et al. (2011). The ileum samples were embedded in paraffin and were mounted on slides. All histopathological evaluations and readings were performed under the microscope by an experienced veterinary histopathologist.

TABLE 4
List of histological parameters for the evaluation of intestinal lesions (ileum).

| Score | Iléon |
|---|---|
| 0 | Normal appearance. |
| 1 | Epithelial integrity, appropriate presence of inflammatory cells, |
| | absence of necrosis, vascular disorders, abnormal desquamation. |
| | Lymphoid hyperplasia possible, with production of primary follicles. |
| 2 | Mild to moderate lesions: hyperemia of the mucosa and submucosa, |
| | slight desquamation, superficial epithelial necrosis of the mucosa, |
| | moderate heterophilic infiltration of the lamina propria or |
| | submucosa, atrophy or hypertrophy of the mucosa, moderate |
| | hemorrhage. Moderate hyperplasia of mucus-producing cells and |
| | mononuclear cells. |
| 3 | Lesions as hereinbefore, but of major degree. Intense heterophilic |
| | infiltration, as well as epithelial and submucosal gland necrosis, |
| | intense mononuclear crypt hyperplasia, severe hemorrhage, and |
| | hyperplasia of mucus-producing cells. |

### 3-B. Results

### A—Hatchability

TABLE 5
Percentage of hatchlings in the in ovo vaccinated group
with respect to the unvaccinated groups (negative
control, positive control, commercial vaccine).

| Group | Hatchability (n = 60) |
|---|---|
| Negative control | 81.2% |
| Positive control | |
| Commercial vaccine | |
| VXN in ovo | 78.3% |

Hatchability was measured in this study to assess the safety of the in ovo VXN/E. coli vaccine. Thus, the hatchability of vaccinated eggs (n=60) was compared with that of unvaccinated eggs (n=180), prior to randomization into each group (negative control, positive control and commercial vaccine). A similar percentage of hatchlings was observed between vaccinated (78.3%) and unvaccinated (81.2%) eggs. As a result, the vaccine formulation is safe because it has no impact on hatchability.

B—Bird Survival after Lethal Challenge

The E. coli challenge was performed at D14 with 4.2×10¹² CFU and directly in the air sacs. Bird survival after lethal challenge is depicted in FIG. 8. This high dose had an impact on bird survival, with 26% mortality observed in unvaccinated birds. Furthermore, mortality increased to 36% in birds vaccinated with the commercial mucosal vaccine, suggesting that it did not induce protection against lethal E. coli infection. On the other hand, it was only 10% for birds vaccinated in ovo with the VXN/E. coli vaccine, suggesting better protection against infection.

C—Quantification of E. coli in Air Sacs by MPN

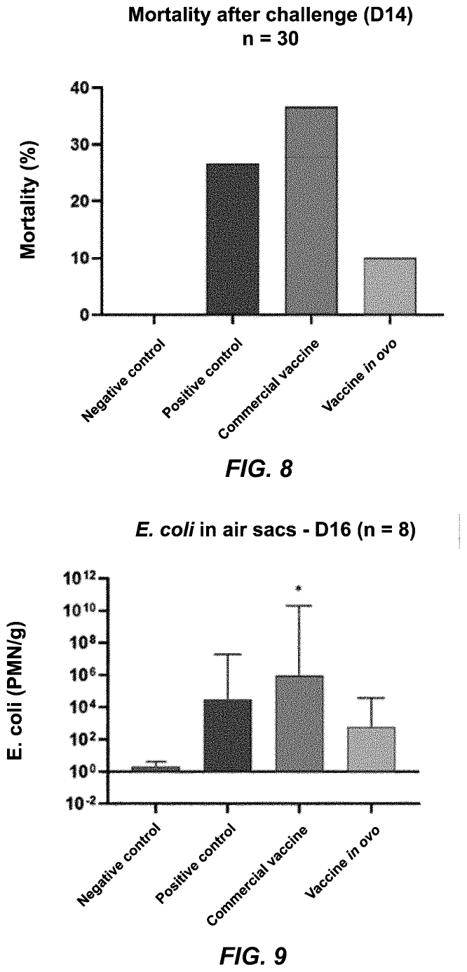

Infection was evaluated by quantifying bacteria in air sacs, by MPN. The measurement of bacterial load in air sacs by MPN is shown in FIG. 9. The birds in the negative control showed only a small amount of bacteria in the air sacs, relative to the natural bacterial flora. Non-immunized challenged birds had higher levels of E. coli (10⁴ MPN/g), which confirms the efficacy of the challenge. Significantly higher infection was observed in the air sacs of birds vaccinated with the commercial mucosal vaccine (10⁶ MPN/g), corroborating the survival results. However, as with the survival results, lower infection was observed in birds vaccinated in ovo with the E. coli/LNP vaccine (3×10³ MPN/g), confirming its efficacy in protecting against this bacterial infection.

### D—Intestinal Permeability

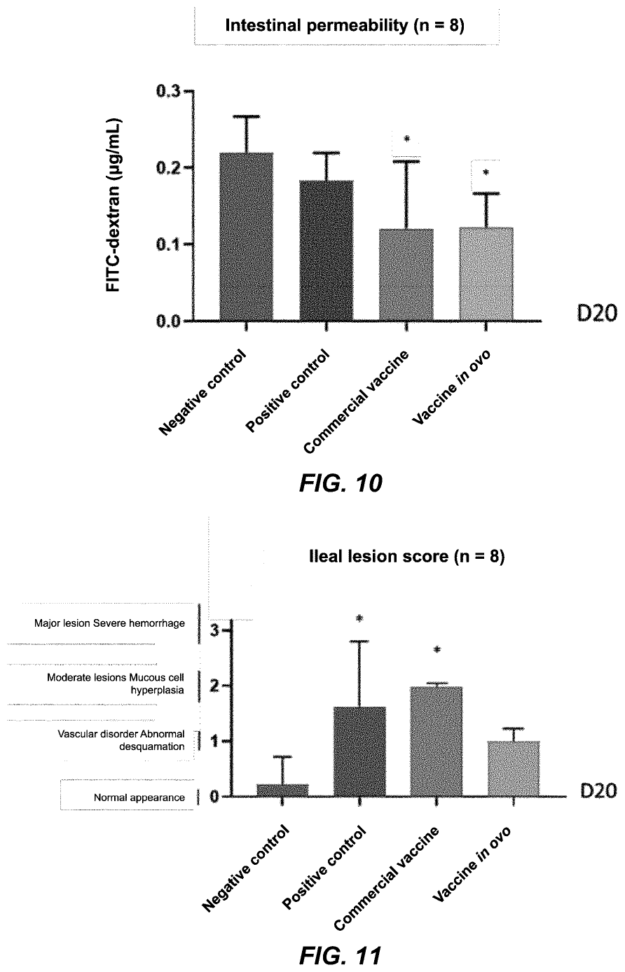

The intestinal permeability for unchallenged, unvaccinated birds was 0.22 μg/mL, and 0.18 μg/mL for challenged, unvaccinated birds. The intestinal permeability is depicted in FIG. 10. When vaccinated with the commercial vaccine or in ovo with the VXN/E. coli vaccine, permeability decreased significantly to 1.2 μg/mL, which suggests vaccine-induced protection.

### E—Intestinal Histopathology

The ileal lesion score was measured 6 days after the challenge. The intestinal lesion score is depicted in FIG. 11. Birds in the negative control group obtained a mean score of less than 1 (0.25), representing a healthy ileum with a normal appearance, as expected. In contrast, unvaccinated birds in the positive control group had a significantly higher mean score of 1.7, suggesting ileal lesions with vascular disorders and desquamation, induced by E. coli infection. Surprisingly, when the birds were vaccinated with the commercial vaccine, the lesions worsened significantly with a mean score of 2. In contrast, birds vaccinated in ovo had a mean score of 1, suggesting protection against E. coli-induced intestinal lesions.

### Conclusion:

This second trial shows that the in ovo VXN-E. coli vaccine protects birds from mortality induced by lethal E. coli infection. Furthermore, this vaccine reduces the bacterial load in air sacs and intestinal lesions caused by infection, confirming the value of this vaccine.

### Example 4: Intramuscular Salmonella Vaccination Trial on Laying Hens

### 2-A. Materials and Methods

### A—Preparation of Vaccines

The vaccine is manufactured from an inactivated strain of Salmonella enteritidis mixed with lipidated maltodextrin nanoparticles (LNP). In brief, Salmonella strain SE147 was inactivated and the protein content was measured by a μBCA assay. Finally, 200 μg of killed bacteria were mixed with either LNP (formulation named “Vaxinano 1”) or non-crosslinked LNP (formulation named “Vaxinano 2”), at a rate of 200 μg of protein per dose of vaccine. Non-crosslinked LNP is composed of linear cationic maltodextrin with an anionic inner core.

### B—Animals

For this experiment, a total of 84 LSL chickens (from a commercial breeding farm) were randomly assigned to 12 pens (7 birds/pen) as described in Table 7. The serum was collected and tested for the Salmonella antibody titer (performed by DGZ using the BioChek kit).

TABLE 6
Identification of experimental groups.

| Group (7 animals/group) | Description |
|---|---|
| 1, 5, 9 | Vaccinated with saline solution |
| 2, 6, 10 | Vaccinated with 200 μg |
| | of “Vaxinano 1” |
| 3, 7, 11 | Vaccinated with 200 μg |
| | of “Vaxinano 2” |
| 4, 8, 12 | Vaccinated with a commercial |
| | vaccine (Salenvac) |

### C—Vaccination

At the age of W12, all chickens were vaccinated by IM in the breast with either 500 μL of a saline solution, or 500 μL of Vaxinano 1 or Vaxinano 2 formulation (containing 200 μg of Salmonella protein), or with the commercial vaccine. One month later, at W16, the animals received a second dose of the same vaccine formulation.

### D—Challenge

One month after the booster, all animals were challenged intravenously with 500 μL of 1.3×10⁸ CFU of S. enteritidis SE147.

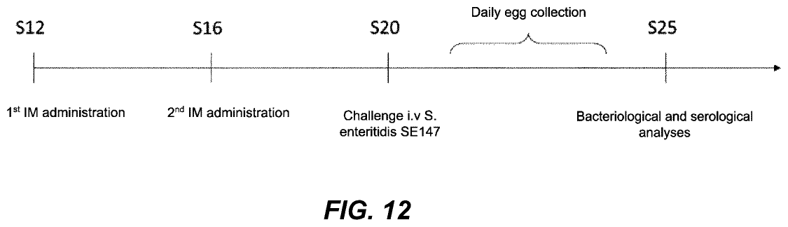

The general schedule is depicted in FIG. 12.

### E—Samples

From W20 to W25, the eggs were collected and bacteriologically analyzed individually for Salmonella. At W25, all chickens were euthanized, and the serum and liver were collected and stored at −20° C. The spleen and cecum were bacteriologically analyzed for Salmonella.

### F—Analyses

Laying: eggs were collected daily after the challenge (except Saturday) and stored at 4° C. The number of eggs per group was reported.

Infection: infection was quantified by qPCR in the spleen and cecum of each chicken at W25.

### 2-B. Results

A—Egg laying

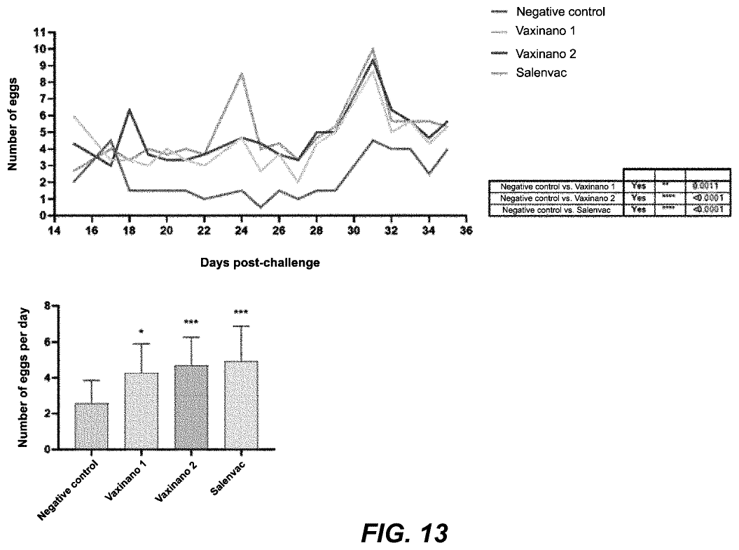

The mean number of eggs laid in each group was counted each day after the test. Egg laying is depicted in FIG. 13. Hens vaccinated with a dummy vaccine laid a low number of eggs after the challenge, with a mean of 2.6 eggs per day, confirming the bird's infection. On the contrary, birds vaccinated with Salenvac laid significantly more eggs than the control group, with a mean of 4.9 eggs per day (p<0.001), which suggests protection against the challenge. Similarly, both Vaxinano formulations enabled hens to lay significantly more eggs, with a mean of 4.3 eggs per day for Vaxinano 1 (p<0.05) and 4.7 for Vaxinano 2 (p<0.001), suggesting equivalent protection against the challenge.

### B—Infection of the Cecum and Spleen

Infection was evaluated by quantifying the bacterial load in the cecum and in the spleen.

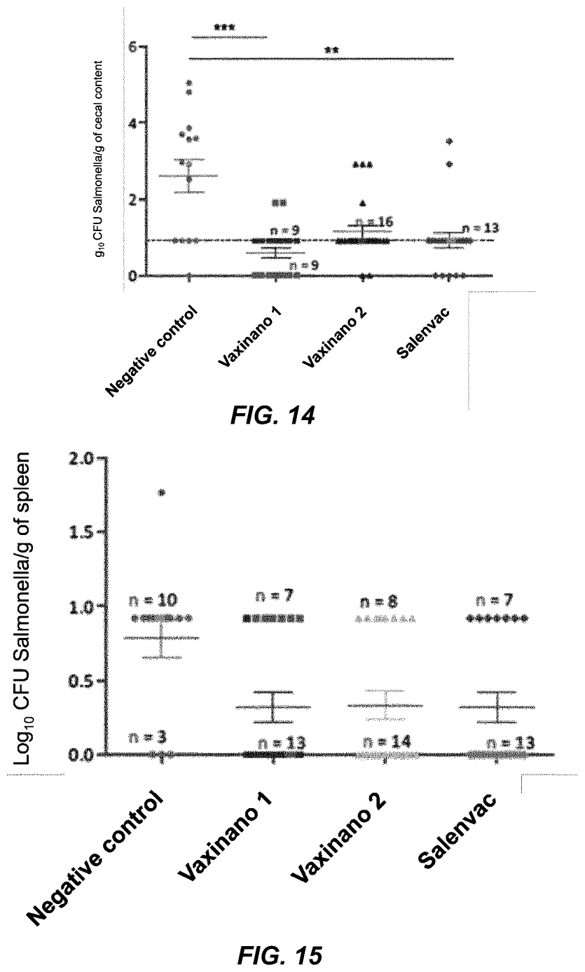

Post-challenge infection was evaluated by quantification of bacterial load in the cecum and in the spleen. This quantification is depicted in FIG. 14. Chickens injected with saline had significant infection, with a mean infection of 400 CFU/g, but with over 50% of birds above 1000 CFU/g. In contrast, all birds vaccinated with either the Vaxinano formulation or with Salenvac had significantly lower infection, with mean infection less than the threshold for birds vaccinated with Vaxinano 1. This confirms the strong protection provided by IM vaccination.

The infection was finally quantified in the spleen. This quantification is depicted in FIG. 15. Although infection was lower than in the cecum, 77% of birds vaccinated with the saline solution were still positive in the spleen, compared with only 35% of birds vaccinated with Vaxinano 1, 36% of birds vaccinated with Vaxinano 2 and 35% of birds vaccinated with the commercial vaccine, confirming the protection provided by IM vaccination.

### Conclusion:

This trial indicates that the inactivated whole Salmonella/NP vaccine administered intramuscularly protects birds from challenge by S. enteritidis, whatever the formulation, enabling birds to lay significantly more eggs than non-immunized animals.

### Example 5: Mucosal Vaccination Against Colibacillosis

This trial was planned in the same way as the in ovo trial. The analyses then focused on the protection provided by vaccines against infection in target organs, physiological abnormalities and mucosal antibody titers.

### 5-A Materials and Method

### A—Preparation of the Vaccine

The vaccine is manufactured from 3 strains of inactivated E. coli bacteria, mixed with lipidated maltodextrin nanoparticles (LNP). Strains O78: K80, O1: K1, O2: K1 were inactivated with 0.4% formaldehyde and the protein content was measured by BCA assay. Finally, 33.3 μg per strain were then mixed with LNP at a rate of 100 μg of protein per vaccine dose.

### B—Animals

All animal work has been reviewed and approved by the Imunova Análises Biológicas Animal Research Ethics Committee, protocol number 06/2021.

For this experiment, 150 day-old chicks were acquired from a commercial hatchery, and randomly sorted into five experimental groups, in individual isolation units within Imunova, and were treated in accordance with the following table:

TABLE 7
Identification of experimental groups.

| Group (30 animals/group) | Challenge¹ |
|---|---|
| Negative control | Unvaccinated, unchallenged |
| Positive control | Unvaccinated, challenged² |
| Commercial vaccine | POULVAC ® E. coli vaccine (mucosal), |
| | challenge² |
| VXN mucosal vaccine | VXN mucosal vaccination, challenge² |
| VXN vaccine s.c | VXN SC vaccination, challenge² |

¹All groups, including the negative control, were infected with the live attenuated Mass I - H120 infectious bronchitis virus (IBV) vaccine orally, at a dose 100x that recommended by the manufacturer, in order to sensitize the animals to the E. coli challenge.
²Infection with 108 CFU of Escherichia coli, at a dose of 100 μL/bird orally. Infection was confirmed by microbiological recovery of bacteria from the inoculum.

### C—Vaccination

Animals in the “VXN Vaccine SC”, “VXN Mucosal Vaccine” and “Commercial Vaccine” groups (POULVAC® E. coli, Zoetis) received a primary immunization at D1 and a second dose at D12. For the “VXN Mucosal Vaccine,” doses were administered in the eye, beak and nostril, while for the “commercial vaccine,” doses were administered in drinking water.

### D—Challenge

The challenge strain was a field isolate confirmed as an APEC by PCR identification of 5 pathogenicity genes (iuaT, iroN, ompC, iss, hly). It was also verified as belonging to phylogroup F by the typing method described by Clermont (Clermont et al., 2013). All animals received a 100× dose of attenuated IBV vaccine (Massachusetts H-120 strain, MASS® I, Zoetis) at D14. The E. coli challenge was carried out in all groups except in the “negative control,” and 10⁸ CFU/bird were used.

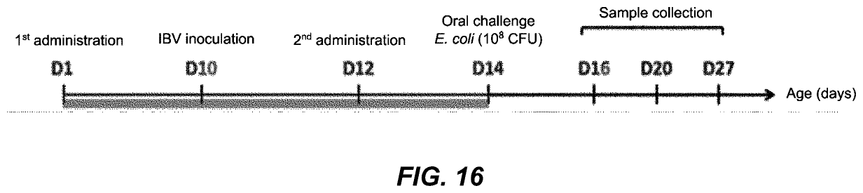

The overall protocol schedule for the in ovo vaccination trial is shown in FIG. 16.

### E—Analysis

Quantification of Anti-E. coli IgA:

The production of specific anti-E. coli IgA was evaluated by ELISA. Briefly, the samples were diluted in 1% casein in PBS. The ELISA plates were coated with E. coli LPS (field isolate strain). The plates were then washed three times with 200 μL/well of PBS+0.05% TWEEN®-20 for 5 min/wash. The wells were blocked with 1% casein in PBS. The samples were tested by serial dilution. The plates were washed and anti-chicken IgA was added (Bio-Rad) diluted in 0.1% casein. After washing, the test was revealed with a single solution of TMB (Life Technologies). Absorbance was read at 450 nm and quantification was performed using a proprietary methodology/kit developed by Imunova.

Quantification and Detection of E. coli Infection:

Detection of E. coli by MPN (most probable number) was carried out on the basis of standard ISO 7251:2005. E. coli counts were determined according to standard microbiological methods (dilution in enrichment medium followed by plating in selective/differential medium). Briefly, samples were enriched in buffered peptone water (BPW), followed by EC broth and finally plating in EMB and MacConkey agar. The samples were serially diluted in triplicate in BPW prior to incubation, to enable quantification by the MPN technique (Blodgett et al., 2015). In the detection of E. coli, only the serial dilution step was omitted. Suspect isolated colonies were biochemically tested and confirmed.

### Histology of the Lungs:

The birds were euthanized and lung samples were taken and fixed. The samples were embedded in paraffin and mounted on slides. All histopathological evaluations and readings were performed under the microscope by an experienced veterinary histopathologist.

TABLE 8
List of histological parameters for evaluating lung damage scores

| Score | Lungs |
|---|---|
| 0 | Normal appearance |
| 1 | Hyperemia. Parabronchial edema. Luminal parabronchial fibrinous |
| | material and endobronchial desquamation. Mononuclear and |
| | heterophilic luminal and mucosal infiltration. Discreetly |
| | compromised aeration. |
| 2 | Hyperemia. Obliterative bronchiolitis with mucofibrinous exudation |
| | and infiltration to a lesser degree by inflammatory, heterophilic and |
| | mononuclear cells. Secondary bronchial disease. Atrial enlargement. |
| | Reasonably maintained aeration. |
| 3 | Hyperemia, perivascular edema. Tertiary bronchial or parabronchial |
| | damage is fairly extensive, with diffuse heterophilic infiltration. The |
| | anterior bronchioles show lymphopurulent peribronchitis and |
| | endobronchitis. Reasonably maintained aeration. |
| 4 | Hyperemia. The primary bronchus shows fibrino-necrotic/purulent |
| | exudation with the epithelial remnant of the desquamated mucosa |
| | showing intense vacuolization. Inflammatory material in the lumen, |
| | associated with necrosis products and without significant aeration of |
| | the most affected air capillaries. Infiltration by mononuclear |
| | heterophils, including macrophages. |

### 5-B: Results

Presence of E. coli in the Airways (MPN)

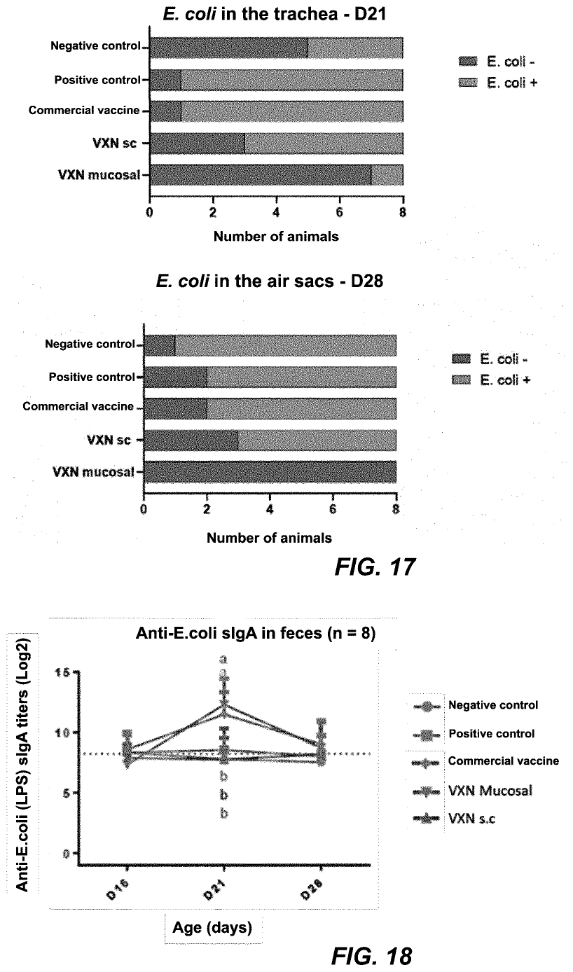

Infection was evaluated by quantifying the number of birds infected in the trachea and in the air sacs, per MPN. The measurement of bacterial infection in the trachea and alveoli is shown in FIG. 17. Three unchallenged birds were infected in the trachea and seven in the air sacs, probably due to the presence of naturally occurring pathogenic E. coli in the environment. On the contrary, unvaccinated challenged birds were more infected in the trachea confirming the efficacy of the challenge. The same number of birds were infected in the group receiving the commercial vaccine with respect to the infected control group, suggesting a lack of protection. However, from the birds vaccinated with the VXN mucosal vaccine, only one bird was infected in the trachea and none in the air sacs. This confirms that the mucosally administered VXN E. coli vaccine protected the birds from the challenge.

Presence of Anti-E. coli sIgA in Feces

E. coli anti-LPS secretory IgA was analyzed in feces by ELISA. The results of the anti-E. coli sIgA analysis are depicted in FIG. 18. From D16 to D28, the antibody titer of birds in the negative control was the same as that in the positive control, indicating that the oral challenge did not induce intestinal IgA secretion. However, for mucosally vaccinated birds, both with the VXN E. coli formulation and with the commercial vaccine, a significant increase in antibody titers was observed at D21. The mucosal vaccine was thus able to induce a humoral response in the intestine.

### Lung Clinical Score

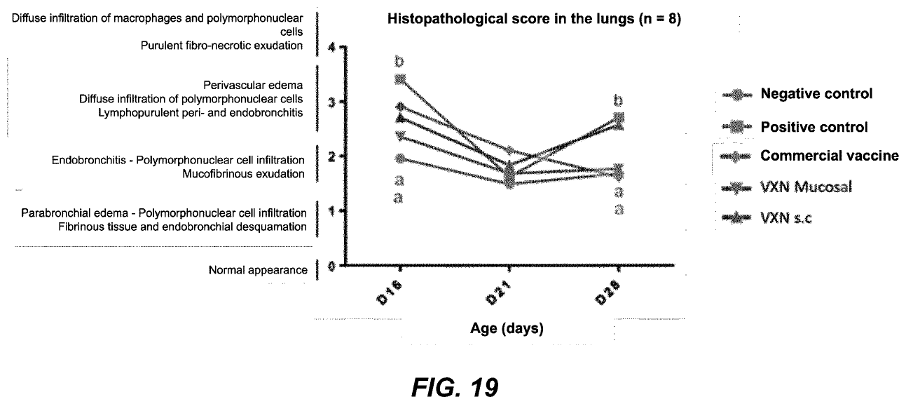

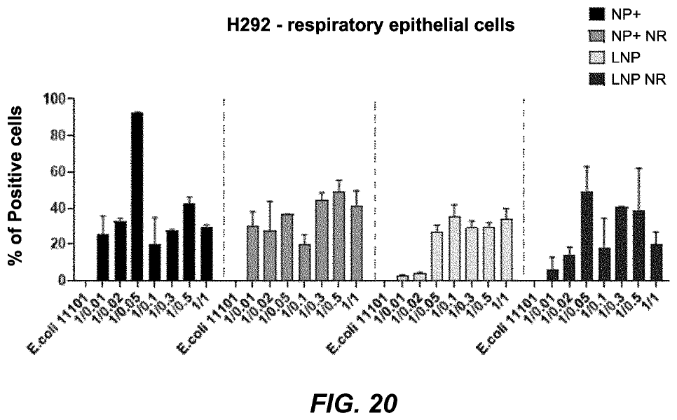

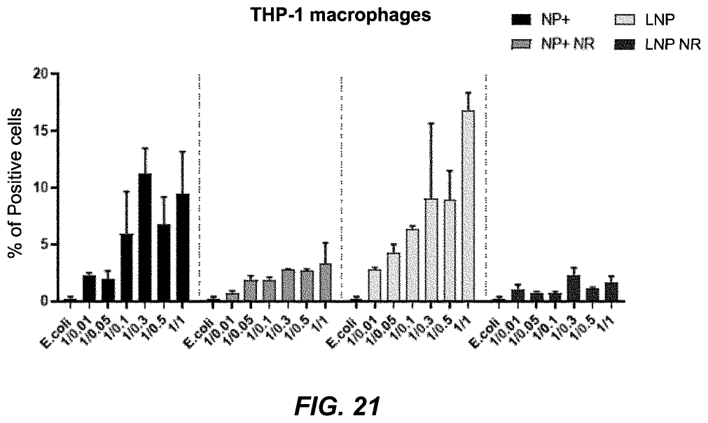

Lung lesion scores were measured from D16 to D28. The lesion scores are depicted in FIG. 19. Although they had the lowest histopathological score from D16 (score=2) to D28 (score=1.7), birds in the negative control group showed endobronchitis, mucofibrinous exudation and polynuclear neutrophil infiltration, which is probably related to natural infection (FIG. 17). Furthermore, unvaccinated challenged birds in the positive control group had the highest score, 3.4 at D16 and 2.7 at D28, due to E. coli challenge. In contrast, birds vaccinated mucosally with the VXN-E. coli formulation had a mean lesion score of 2.3 at D16, less than the commercial vaccine (score=2.9). Finally, at D28, both groups of mucosally vaccinated birds had a score comparable to the negative control (1.6 for the commercial vaccine, 1.8 for the VXN vaccine), suggesting protection against lung lesions induced by E. coli infection.

### Example 6: Optimizing the Formulation with Different Cationic Particles

A study was carried out on an inactivated whole E. coli strain, to compare formulations prepared with different maltodextrin-based particles, measuring their uptake by immune cells in vitro.

### 6-A Materials and Methods

### A—Vaccine Formulations

The vaccine formulations were made herein with one of the inactivated E. coli strains used in the vaccine (11101), mixed with maltodextrin (NP+) or lipidated nanoparticles (LNP), as well as cationized but non-crosslinked, single (NP+NR) or lipidated maltodextrin (LNP-NR). The nanoparticles partially to very partially cover the bacteria.

### B—Particle Synthesis and Characterization

NP+ are nanoparticles synthesized from cationic and crosslinked maltodextrin. More specifically, the synthesis consists of maltodextrin (Roquette, France) dissolved in a 2M NaOH solution under magnetic stirring and at room temperature. Epichlorohydrin (Merck group, France) was then added as a crosslinking agent, along with glycidyl trimethylammonium (GTMA, Merck group, France) as a cationizing agent. The resulting gel was then neutralized with acetic acid and ground in a very high-pressure homogenizer (LM20, Microfluidics, France). The crushed material was then purified by tangential flow filtration (AKTA flux 6, GE Healthcare, France) through a 750 kDa membrane (GE Healthcare, France) to obtain purified NP+. These NP+ are capable of encapsulating antigens from various pathogens (viruses, bacteria or parasites) and of delivering them to immune cells (1).

Similarly, LNPs are NP+ wherein a core of anionic phospholipids (DPPG) has been added. More specifically, a solution of dipalmitoyl phosphatidylglycerol (DPPG, Lipoid, Germany) is dissolved in solutol, then injected into an NP+ solution with stirring, at a mass percentage of 70%. The phospholipids are incorporated into the core of the nanoparticle, thus forming LNPs. These LNPs are also capable of encapsulating antigens from various pathogens (viruses, bacteria or parasites) and of delivering them to immune cells (2-4).

Finally, NP+NR and LNP-NR are their respective equivalents, synthesized according to the same synthesis scheme but without crosslinking agents, thus forming linear cationic polymers.

The particles were characterized according to their size by dynamic light scattering (DLS) and according to their surface charge (or zeta potential) by electrophoretic light scattering (ELS), using a Zetasizer Nano ZS (Malvern, France).

C—Inactivation of E. coli Bacteria

The bacteria were inactivated with 0.4% formaldehyde, then purified by centrifugation. The protein content of whole bacteria was measured by micro BCA assay. The formulation was made by mixing the killed bacteria with an aqueous particle solution, at different weight ratios (100 μg of E. coli protein with 1, 5, 10, 30, 50 or 100 μg of particulate).

D—Delivery of E. coli into Cells by the Particles

The ability of the particles to enhance phagocytosis-mediated uptake of inactivated whole E. coli by epithelial cells and into macrophages was evaluated by flow cytometry.

Labeling Proteins with Fluorescein:

Inactivated E. coli were labeled with fluorescein isothiocyanate (FITC), by mixing 5 mg of E. coli with 50 μg of FITC (1% w/w, Merck, France) in sodium carbonate buffer at pH 8.3 for 2 hours. They were then dialyzed on a 10 kDa dialysis cassette (Thermo Fisher, France). The protein content of whole bacteria was measured by micro BCA assay (Pierce, France). The labeled bacteria were then associated with particles at different weight ratios.

### E—Flow Cytometry:

H292 cell lines were seeded in 24-well plates at 50,000 cells per well, and processed after 3 days of culture. THP-1 cell lines were seeded in 24-well plates at 100,000 cells per well, and differentiated into macrophages with 20 ng/ml of PMA for 24 hours. After changing the culture medium, the cells were incubated with the equivalent of 1 μg of killed bacteria, alone or combined with particles, for 4 hours. The cells were then washed with phosphate-buffered saline (PBS), harvested with trypsin, and analyzed by flow cytometry on an Attune Nxt (Thermo Fisher, France).

### 6-B Results

### Particle Characterization

TABLE 9
Characterization of the size of different particles (Z-average
and Number) and their surface charge (zeta potential).

| | Z-Average (nm) | Number (nm) | Zeta potential (mV) |
|---|---|---|---|
| NP⁺ | 92 ± 7 | 33 ± 4 | 36 ± 5 |
| NP⁺NR | 50 ± 3 | 17 ± 3 | 32 ± 2 |
| LNP | 83 ± 15 | 36 ± 5 | 39 ± 2 |
| LNP-NR | 54 ± 12 | 23 ± 9 | 38 ± 3 |

The physicochemical characteristics of the different particles were analyzed after their synthesis (Table 9). NP+ had a diameter of 33 nm and a surface charge of 36 mV, and LNP had a diameter of 36 nm and a surface charge of 39 mV, indicating that the phospholipids were associated with the particles not on their surface but within their maltodextrin structure.

NP+NRs had a diameter of 17 nm for a surface charge of 32 mV, and LNP-NRs had a diameter of 23 nm with a surface charge of 38 mV, similarly indicating that the phospholipids were associated within the maltodextrin structure.

In the absence of crosslinking agent, the particles obtained appeared to have a smaller diameter. Indeed, without a crosslinking agent, maltodextrin in solution should remain predominantly linear, but it could also fold in on itself through hydrophobic bonds, thus forming nanoparticles detected during analysis.

Phagocytosis of E. coli by Cells after Association with Particles

Delivery of E. coli by the particles was first evaluated on respiratory epithelial cells (H292). Without particles, the bacteria were not endocytosed by the cells (FIG. 2). When they were covered by the particles, their uptake greatly increased, reaching 20-50% of positive cells, whatever the particles used. Furthermore, improved phagocytosis could be observed as early as the 1:0.01 ratio, which demonstrates that a small amount of particles partially covering the bacteria is sufficient to improve intracellular delivery.

Similarly, E. coli phagocytosis by the particles was evaluated on differentiated macrophages (THP-1). Without particles, bacteria were phagocytized by 0.2% of cells (FIG. 3). When they were covered by the particles, their phagocytosis increased to 4-15% of positive cells depending on the ratios. Furthermore, improved phagocytosis could be observed as from the 1:0.01 ratio, which demonstrates once again that a small amount of particles covering the bacteria is sufficient to improve bacterial delivery.

### CONCLUSION

Bacterial adhesion and uptake by respiratory epithelial cells and by macrophages can be enhanced by single (NP⁺) or lipidated (LNP) cationized maltodextrin nanoparticles, as well as by single (NP⁺NR) or lipidated (LNP-NR) non-crosslinked cationic maltodextrin, equivalently between NPs. A low dose of particles (10 to 100 times fewer particles than bacteria in mass %) is sufficient to improve this delivery to cells.

1. An adjuvant-free vaccine composition comprising cationic nanoparticles having a polysaccharide core and at least one inactivated bacterium, wherein the bacterium is whole and the cationic nanoparticles cover the bacterium.

2. The vaccine composition of claim 1, wherein the cationic nanoparticles (NP) cover the bacterium in a bacterial protein:NP ratio greater than or equal to 1 by weight.

3. The vaccine composition of claim 2, wherein the bacterial protein:NP ratio is greater than or equal to 5 by weight.

4. The vaccine composition of claim 1, wherein the cationic nanoparticles have a non-lipid-loaded, crosslinked polysaccharide core comprising (i) a polysaccharide selected from the group consisting of starch, dextran, chitosan, dextrin, maltodextrin, polyfructoses, polymannoses, polygalactoses, and polygalactomannans, (ii) at least one cationic ligand selected from the group consisting of a primary, secondary or tertiary amine and quaternary ammoniums, and (iii) a crosslinking agent selected from the group consisting of epichlorohydrin, a dicarboxylic acid, and an acid chloride.

5. The vaccine composition of claim 1, wherein the cationic nanoparticles have a non-crosslinked, non-lipid-loaded polysaccharide core comprising (i) a polysaccharide selected from the group consisting of starch, dextran, chitosan, dextrin, maltodextrin, polyfructoses, polymannoses, polygalactoses, and polygalactomannans, and (ii) at least one cationic ligand selected from the group consisting of a primary, secondary or tertiary amine and quaternary ammoniums.

6. The vaccine composition of claim 1, wherein the cationic nanoparticles have a phospholipid-loaded, crosslinked polysaccharide core comprising (i) a polysaccharide selected from the group consisting of starch, dextran, chitosan, dextrin, maltodextrin, polyfructoses, polymannoses, polygalactoses, and polygalactomannans, (ii) at least one cationic ligand selected from the group consisting of a primary, secondary or tertiary amine, and quaternary ammoniums, (iii) a crosslinking agent selected from the group consisting of epichlorohydrin, a dicarboxylic acid, an acid chloride, and (iv) an anionic phospholipid selected from the group consisting of diacyl phosphatidyl glycerol, diacyl phosphatidyl serine, and diacyl phosphatidyl inositol.

7. The vaccine composition of claim 1, wherein the cationic nanoparticles have a phospholipid-loaded, non-crosslinked polysaccharide core comprising (i) a polysaccharide selected from the group consisting of starch, dextran, chitosan, dextrin, maltodextrin, polyfructoses, polymannoses, polygalactoses, and polygalactomannans, (ii) at least one cationic ligand selected from the group consisting of a primary, secondary or tertiary amine, and quaternary ammoniums, and (iii) an anionic phospholipid selected from the group consisting of diacyl phosphatidyl glycerol, diacyl phosphatidyl serine, and diacyl phosphatidyl inositol.

8. The vaccine composition of claim 4, wherein the polysaccharide core comprises maltodextrin, and a glycidyl trimethylammonium.

9. The vaccine composition of claim 6, wherein the polysaccharide core is loaded with anionic phospholipid selected from the group consisting of diacyl phosphatidyl glycerol, diacyl phosphatidyl serine, and diacyl phosphatidyl inositol.

10. The vaccine composition of claim 1, wherein the at least one inactivated bacterium comprises at least two different strains of bacteria.

11. The vaccine composition according to claim 1, wherein the at least one inactivated bacterium comprises a bacteria selected from the group consisting of: Salmonella enterica ser. Typhi; Streptococcus pneumoniae; Haemophilus influenzae type b; Mycobacterium tuberculosis; Extraintestinal pathogenic E. coli (ExPEC); enterotoxigenic E. coli (ETEC); S. enterica ser.; Paratyphi A; Neisseria gonorrhoeae; Clostridium difficile; Campylobacter spp; Shigella spp; Staphylococcus aureus; and Helicobacter pylori.

12. A method of vaccinating a mammal or bird, the method comprising administering to the mammal or bird or an embryo thereof a composition comprising cationic nanoparticles having a polysaccharide core and at least one inactivated bacterium, wherein the bacterium is whole and the cationic nanoparticles cover the bacterium, the administering comprising intramuscular, mucosal or in ovo administration.

13. A method of preventing a bacterial infection in a mammal or bird, the method comprising:
administering to the mammal or bird the vaccine composition of claim 1 so as to prevent the bacterial infection in the mammal or bird.

14. A method of preventing salmonellosis, colibacillosis or campylobacteriosis in a subject, the method comprising:
administering to the subject the vaccine composition of claim 1 so as to prevent the salmonellosis, colibacillosis or campylobacteriosis in the subject.

15. The method of claim 12, wherein the administering comprises in ovo administration.

16. The vaccine composition of claim 5, wherein the polysaccharide core comprises maltodextrin and a glycidyl trimethylammonium.

17. The vaccine composition of claim 6, wherein the polysaccharide core comprises maltodextrin and a glycidyl trimethylammonium.

18. The vaccine composition of claim 7, wherein the polysaccharide core comprises maltodextrin and a glycidyl trimethylammonium.

19. The vaccine composition of claim 7, wherein the polysaccharide core is loaded with anionic phospholipid selected from among the group consisting of diacyl phosphatidyl glycerol, diacyl phosphatidyl serine or diacyl phosphatidyl inositol.
